(19) United States
(12) Patent Application Publication
(10) Pub. No.: US 2011/0262486 A1
(43) Pub. Date: Oct. 27, 2011

(54) Bone implant and manufacturing method thereof

(75) Inventors: Yu-Hui Tsai, Banciao City (TW); Li-Hsuan Chiu, Taipei City (TW); Wen-Fu Lai, Taipei City (TW); Shih-Ching Chen, Taipei City (TW)

(73) Assignee: TAIPEI MEDICAL UNIVERSITY, Taipei City (TW)

(21) Appl. No.: 12/799,343

(22) Filed: Apr. 22, 2010

(51) Int. Cl. A61K 9/00; A61K 33/24; A61K 35/12; A61K 38/39

(52) U.S. Cl. 424/400; 424/617; 424/93.7; 514/8.1; 514/8.2; 514/8.6; 514/8.7; 514/17.2; 514/8.9; 514/9.1

(57) ABSTRACT

The invention discloses a bone implant and a manufacturing method thereof. The manufacturing method of the bone implant comprises a step of coating or mixing type II collagen with at least one porous bone material comprising metals, bio-ceramics, natural biopolymers and synthetic polymers. Another manufacturing method of the bone implant comprises the steps of loading type II collagen with or without at least one porous bone material in a container, and lyophilizing the type II collagen to generate a type II collagen sponge construct with or without the porous bone material as the bone material. The manufactured bone implants are effective, with or without loading cells having differentiation tendency towards osteogenesis, to facilitate bone repair upon introduction of the bone implant into various osseous defects.

### FIELD OF THE INVENTION

The present invention relates to a field of bone regeneration, and in particular to a bone implant and a manufacturing method thereof.

### BACKGROUND

Bone is a hardened connective tissue composed of cells and extracellular matrices (ECMs). Different from other connective tissue, the matrices of bone tissue are mineralized. In human body, it is an excessively hard tissue for providing support for body weight and load, and protection from physical stress. In case that bone tissue fails to repair itself at a normal rate, or that bone loss occurs as a result of injuries or diseases, it may lead to disability and huge waste of time and money. Osteogenesis, the growth of new bone, is a part of the normal healing process which involves the recruitment and activation of osteoblasts and mesenchymal stem cells (MSCs). In the elderly, osteogenesis after disease or severe trauma can be a slow process. Therefore, after trauma and orthopaedic or dental procedures, accelerating osteogenesis and speeding the healing process are an important issue in this field.

Endochondral ossification is one of the two essential processes during osteogenesis and fetal bone development. Unlike intramembranous ossification, which dominates the rudimentary formation of cranium, cartilage is present during the endochondral ossification process. Endochondral ossification presents as an essential process during the embryonic development of long bones and the natural healing of fractures. It is a highly coordinated, multi-step process. Briefly, cascades of events participated in this developmental process include MSC condensation, chondrocyte differentiation/maturation/hypertrophy, cartilage template mineralization, and the consequent invasion and differentiation of the osteo-progenitor cells. Extracellular matrix components have also been demonstrated to play crucial roles in coordinating and directing MSC differentiation. This interaction triggers the differentiation of osteo-progenitors and leads to the mineralization of the tissue into mature bone structure.

Bone implantation may be necessary in the damage of the bone owing to the fracture, trauma, or the pathological causes. Implants are commonly used in the medical profession to replace or reinforce the injured or diseased hard bones. Besides, there have been a lot of materials and substances used in bone repair to regenerate the defective or missing bone. These studies have been undertaken in the effort to activate bone formation at the site in need of bone replacement. As is known, type I collagen enhances osteogenesis of human MSCs and osteoblasts. Upon their attachment to the type I collagen-coated surface, the osteogenic differentiation of these cells can be stimulated via an ERK1/2 signaling pathway. For this reason, the existing application of type I collagen is mostly to mix type I collagen with calcium phosphate to fabricate scaffolds as bone filling material. However, no report has addressed on the modulating effect of type II collagen in promoting osteogenesis. Type II collagen is mainly presented in the cartilage as well as in the developing bone, and is largely considered as a cartilaginous ECM in previous studies. Therefore, type II collagen is rarely discussed about its mechanism and importance in the process of osteogenesis, and is also not being applied to the promotion of bone formation.

### SUMMARY

One aspect of the present invention is to provide a manufacturing method of a bone implant, comprising that at least one porous bone material is coated or mixed with type II collagen, wherein the porous bone material is made of a material selected from the group consisting of metals, bio-ceramics, natural biopolymers and synthetic polymers. Applications of this method comprise in vitro culture of bone cells, auto- and allo-grafts, and bone reconstruction with various bone materials.

According to another aspect of the present invention, a bone implant is provided by the above-mentioned manufacturing method. The bone implant comprises type II collagen and the at least one porous bone material, wherein type II collagen is coated on the surface of the porous bone material, or is mixed with the porous bone material.

Another yet aspect of the present invention is to provide manufacturing method of a bone implant, comprising steps of loading type II collagen with or without at least one porous bone material in a container, and lyophilizing type II collagen to generate a type II collagen sponge construct with or without the porous bone material. Wherein, a concentration of the type II collagen may be in a range of 2 to 20 mg/ml.

The present invention may have one or more advantages as follows:

(1) For the sake of facilitating bone repair and regeneration, type II collagen in the present invention can be used alone, be coated on the surface of the bone material, be mixed with the bone material, or a combination thereof to achieve the desired effect.

(2) Type II collagen according to the present invention can activate ERK1/2 and JNK signaling pathways, thereby elevating the activity of alkaline phosphatase (ALP). Compared to the known type I collagen, the bone implant comprising type II collagen not only can accelerating calcium deposition, but also quickly increase the amount of the bone deposition so as to achieve fast bone regeneration. Therefore, the present invention has the potential for clinical applications to bone repair, or for the development of coated materials used on the surface of the existing bone materials.

(3) After the bone implant prepared according to the present invention is implanted at a site in which bone replacement is required, the osteogenic differentiation can be stimulated and the extent of bone deposition can be greatly increased.

(4) During the healing process, the added type II collagen, either coated on the surface of or mixed with the bone material, may stimulate certain cell populations to form new bone tissue which serve to replace what is lost or damaged. Such type II collagen has potential to be used in clinical situations where skeletal tissue regeneration is necessary to restore normal function, for example, at sites of bone trauma and sites of periodontal defects. In addition, such type II collagen can enhance or promote bone ingrowth into various prosthetic devices and porous bone materials, such as auto- or allo-grafts, processed xenogenic bone chips and the like.

(5) Type II collagen of the present invention can be coated on the scaffold made of various bone materials, or be directly mixed with various bone materials, wherein the bone materials may be the metal, such as titanium or titanium alloy; the bioceramics, such as hydroxyapatite (HA), aluminum oxide, zirconium oxide, calcium sulfate, calcium phosphate, tricalcium phosphate, hydroxyapatite-tricalcium phosphate (HA-TCP) or a combination thereof; the natural polymer, such as alginate, chitosan or a combination thereof; and the synthetic polymer, such as poly-lactic acid (PLA), poly-glycolic acid (PGA) or poly-lactic-co-glycolic acid (PLGA). Accordingly, there is a wide range of applications according to the present invention.

(6) The required type II collagen in the present invention is easily available. For example, type II collagen can be manufactured by genetic recombination of type II collagen cDNA, or by extraction and purification from a cartilage tissue of an animal comprising poultry, livestock or fishes.

### BRIEF DESCRIPTION OF THE DRAWINGS

The exemplary embodiments of the present invention will be understood more fully from the detailed description given below and from the accompanying drawings of various embodiments of the invention, which, however, should not be taken to limit the invention to the specific embodiments, but are for explanation and understanding only.

FIG. 1 illustrates the calcium deposition staining images of MSCs cultured on the non-coated (control), type I collagen-coated (CI-coated), and type II collagen-coated (CII-coated) surfaces in the osteogenic medium for 7, 14, 21 days;

FIG. 2 illustrates the hematoxylin/eosin (H&E) staining images of sliced sections (A) type I collagen sponge scaffold and (B) type II collagen-coated type I collagen sponge scaffold after seeded with MSCs and cultured in the osteogenic medium for 14 days;

FIG. 3 illustrates the calcium deposition level of MSCs cultured on non-coated (control), type I collagen-coated (CI-coated), type II collagen-coated (CII-coated), and 1:1 ration type I/type II collagen mixture-coated (CI+CII-coated) poly-lactic acid (PLA) scaffold in the osteogenic medium for 42 days;

FIG. 4 illustrates the SEM images of 3D cultured MSCs on (B) non-coated, (C) CI-coated, and (D) CII-coated hydroxyapatite-tricalcium phosphate (HA-TCP) scaffolds, and (A) non-cell blank HA-TCP scaffold in the osteogenic medium for 21 days;

FIG. 5 illustrates the SEM images of 3D cultured MSCs on (B) non-coated, (C) CI-coated, and (D) CII-coated hydroxyapatite-tricalcium phosphate (HA-TCP) scaffolds, and (A) non-cell blank HA-TCP scaffold in the osteogenic medium for 42 days.

### DETAILED DESCRIPTION

As used herein, the term “type II collagen” not only refers to type II collagen itself, but also refers to its biologically active fragment and analogue.

As used herein, the processes of the bone repair and regeneration include cell proliferation, cell differentiation, matrix remodeling and angiogenesis.

The present invention provides a manufacturing method of a bone implant, comprising coating or mixing at least one porous bone material with type II collagen, wherein the porous bone material is made of a material selected from the group consisting of metals, bioceramics, natural biopolymers and synthetic polymers. Therefore, a bone implant comprising type II collagen and the porous bone material is obtained by this manufacturing method. The concentration of type II collagen is about 5-1000 μg/ml, preferably 20-200 μg/ml, for coating the readymade implants (i.e. the porous bone material), and 0.1-10 mg/ml for mixing with other porous bone materials to generate the bone implant. The type II collagen may be obtained by genetic recombination of type II collagen cDNA, or by extraction and purification from a cartilage tissue of an animal comprising poultry, livestock or fishes.

Preferably, the metal comprises titanium or titanium alloy; the bio-ceramics comprises hydroxyapatite (HA), aluminum oxide, zirconium oxide, calcium sulfate, calcium phosphate, tricalcium phosphate, hydroxyapatite-tricalcium phosphate (HA-TCP) or a combination thereof; the natural biopolymer comprises alginate, chitosan, collagen, agarose, natural extracellular matrix components, or a combination thereof; and the synthetic polymer comprises poly(lactic acid) (PLA), poly(glycolic acid) (PGA) or poly(lactic-co-glycolic acid) (PLGA), or a combination thereof.

The present invention further provides a manufacturing method of a bone implant, comprising steps of loading type II collagen in a container, and lyophilizing type II collagen to generate a type II collagen sponge construct. Wherein, a concentration of the type II collagen is 2-20 mg/ml, preferably 4-10 mg/ml.

Moreover, a growth factor as a regulator of bone repair and regeneration, such as bone morphogenetic protein (BMP), transforming growth factor-β (TGF-β), fibroblast growth factor (FGF), insulin-like growth factor (IGF-I), vascular endothelial growth factor (VEGF), and platelet derived growth factor (PDGF), is optional to add into the bone implant obtained from the above-mentioned manufacturing methods. Preferably, BMP is BMP-2, and TGF-β is TGF-β1.

Optionally, the stem cells, progenitor cells and osteoblasts may be added into the bone implant obtained from the above-mentioned manufacturing methods. Wherein, the stem cells and progenitor cells have a tendency toward the osteogenic differentiation. Preferably, the stem cells may be mesenchymal stem cells obtained from bone marrow, umbilical cord blood or other somatic tissues, stem cells obtained from baby teeth or permanent teeth, or embryonic stem cells; the progenitor cells may be mesenchymal progenitor cells obtained from bone marrow, umbilical cord blood or other somatic tissues.

### EXAMPLES

The present invention will be better understood by reference to the following Examples, which are provided as exemplary embodiments of the invention, and not by way of limitation.

### Example 1

### The Effect of Type II Collagen on Mesenchymal Stem Cell (MSC) Osteogenesis

In this example, the modulating effects of type II collagen (CII) and type I collagen (CI) on mesenchymal stem cell (MSC) osteogenesis are examined.

MSC Isolation, Cultivation & Storage

Bone marrow aspirates are obtained aseptically from donors (18˜65-year-old) who receive femoral or iliac surgery. Bone marrow is aspirated using a 10 ml syringe. The aspirates are immediately mixed with sodium-heparin, and diluted in five volumes of phosphate-buffered saline (PBS). The cell suspension is then fractionated by overlay on a Percoll gradient (40% initial density, Pharmacia) and centrifuged. The MSC-enriched interface fraction is collected and plated in a 10-cm dish containing 10 ml Dulbecco's Modified Eagles Medium with 1 mg/ml glucose (DMEM/LG, Sigma D5523), 10% FBS, 1× penicillin/streptomycin/fungizone. The medium is changed every four days. When cells reach 80% confluence, they are trypsinized and passaged into new 10-cm dishes at a cell density of 5×10⁵ cells/dish.

Surface Coating

Tissue culture dishes are coated with purified ECM proteins (fibronectin, type I collagen or type II collagen) at a concentration of 5-1000 μg/ml, preferably 20-200 μg/ml, more preferably 20 μg/ml, for 2 hours at room temperature. After incubation, the remaining ECM solution is removed. The collagen-coated dishes are further washed with PBS. The coated dishes are then UV-sterilized and stored at 4° C. till use.

Calcium Deposition Assay Using Alizarin Red S Staining

MSCs are plated on type I collagen (CI)-coated, type II collagen (CII)-coated, or non-coated control culture dishes. After attached, cells are then treated with osteogenic medium, made of 10⁻⁷ M dexamethason, 10 mM β-glycerolphosphate, and 50 μg/ml ascorbic acid in DMEM-LG, to induce osteogenic differentiation. Cells are cultured for day 7, 14 or 21 days. To detect calcium deposition on the cell layer of differentiated MSCs, cells are rinsed rapidly with distilled water. Then, 1 ml of pH 4.2 Alizarin Red S solution is added to cover cell surface for 5 minutes followed by washing thoroughly with distilled water. The calcium deposits exhibit orange red coloration on the cell surface, and are recorded photographically or microscopically. The staining can be further extracted in 10% cetylpyridinium chloride (CPC) and subjected to spectrophotometer detection at 560 nm to quantify the extent of positive staining.

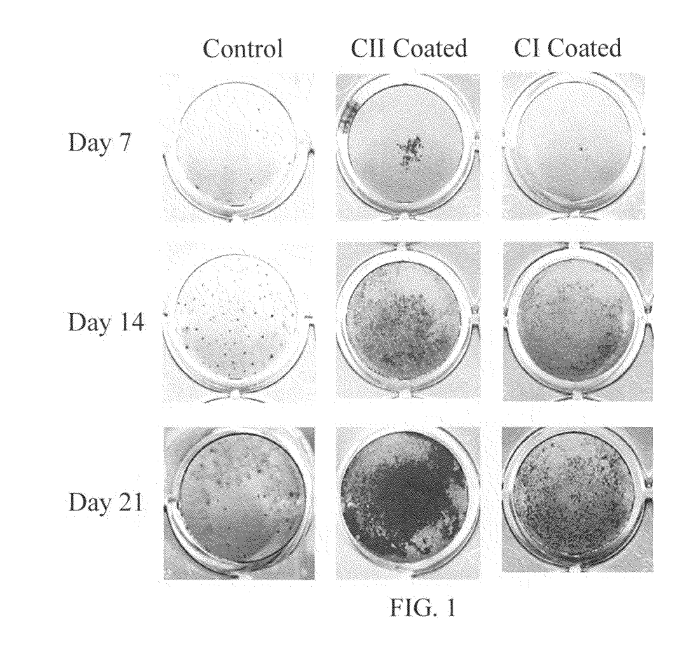

FIG. 1 shows the images of calcium deposition on dishes to illustrate the modulating effects of type II collagen (CII) and type I collagen (CI) on mesenchymal stem cell (MSC) osteogenesis. In the figure, monolayer MSCs are cultured on type II collagen (CII), type I collagen (CI)-coated and non-coated control culture dishes in osteogenic medium for 7, 14 and 21 days. Cells are then fixed and subjected to Alizarin Red S staining for the detection of calcium deposition. Cells in type II collagen-coated groups exhibit an effective mineralization much earlier than that in the type I collagen-coated group and control groups. This result addresses that type II collagen-coated surface accelerates calcium deposition of MSCs in osteogenic medium faster than does type I collagen.

### Example 2

### The Osteogenesis Effect of Type II Collagen Sponge Construct as Bone Implant

In this example, the osteogenic enhancing effects of type II collagen-coated type I collagen scaffold on mesenchymal stem cell (MSC) differentiation is examined.

Fabrication of Three-Dimensional Collagenous Scaffold

Collagens having concentration ranging from 2-20 mg/ml are lyophilized in the 96 well plates. Briefly, 300 μl of type I collagen was loaded in the 96 well plate and lyophilized in a freeze dryer to generate cylinder-like spongy collagenous scaffold as the bone material. After lyophilization, the scaffolds were further coated with type II collagen at a concentration of 5-1000 μg/ml, preferably 20-200 μg/ml, or with 5 mM acetic acid (collagen solvent; as control) for 2 hour at room temperature. After incubation, the remaining solution is removed. The scaffolds were then further washed with PBS and air-dried in the culture hood with UV light on.

MSCs Seeding

Aliquots of 5×10⁵-1×10⁶ human mesenchymal stem cells (MSCs) were suspended in 20 μl of culture medium and loaded into the scaffold. After 2 hours of cell attachment in a 37□ CO₂ incubator, fresh medium was then added to the wells for further cultivation. The cell-loaded collagen scaffold were then transferred to a 24 well plate on the next day, and maintained in designated culture condition for 14 days.

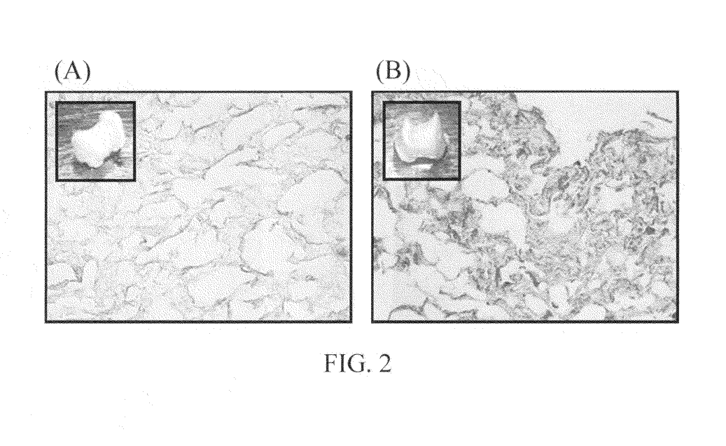

As shown in FIG. 2, type I collagen sponge scaffold (FIG. 2(A)) shows a lighter and thinner H&E staining of the matrices, which indicates the low extent of mineralization in the group. In the type I collagen sponge scaffold with further type II collagen coating (FIG. 2(B)), a heavier and thicker H&E staining is noted, indicating a greater amount of calcified fibrils in the matrices. Upper-left inserts in the images show the respective gross views of the neo-bone derived from MPC-loaded collagen sponge constructs after 14 days of culture. It is concluded that the calcification of MSCs in type I collagen sponge scaffold with further type II collagen coating is greater than that in solely type I collagen-fabricated sponge scaffold.

### Example 3

### The Enhanced Osteogenic Effects of the Type II Collagen-Coated Readymade Bone Materials or Implants

In this example, the osteogenic effects of type I collagen- or type II collagen-coating on poly-lactic acid (PLA) scaffolds/implants are examined.

3D Surface Coating

3D PLA bone scaffolds (herein referring to porous bone materials) are coated with purified ECM proteins (type I collagen or type II collagen) at a concentration of 5-1000 μg/ml, preferably 50-100 μg/ml, or with 5 mM acetic acid (collagen solvent; as non-coating control) for 2 hours at room temperature. After incubation, the remaining ECM solution is removed. The various bone scaffolds are further washed with PBS. The scaffolds are then air-dried in the culture hood under UV light, and stored at 4□ till use.

MSCs Seeding

On aliquots MSCs of 1×10⁵ are suspended in 175 μl of culture medium is seeded into the said PLA bone scaffolds with/without coating collagens on their surfaces. After 2 hours of cell attachment in a 37□ CO₂ incubator, the cell-loaded PLA constructs (herein referring to bone implants of the present invention) are then transferred to a 24 well plate and further cultivated for designated time intervals under osteogenic condition stated above. At the end of designated time intervals, the cell-loaded PLA constructs are subjected to SEM analysis.

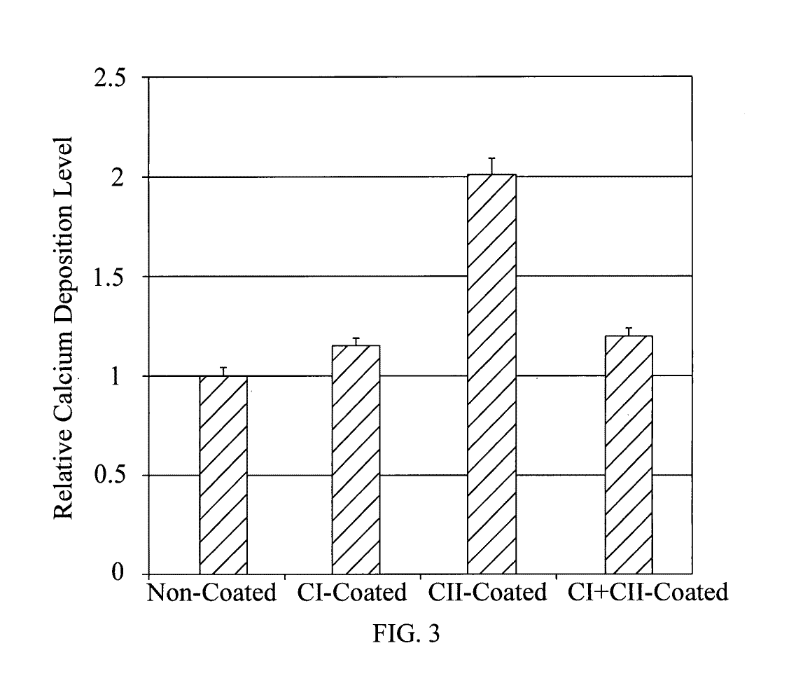

FIG. 3 shows the calcium deposition level of MSCs cultured in PLA scaffolds in osteogenic medium for 42 days. In the figure, cells are cultured on type I collagen-coated (CI-coated), type II collagen-coated (CII-coated), type II and type I collagen (1:1) mixture-coated (CI+CII-coated) and non-coated control PLA scaffold. After 42 days of culture, all scaffolds are fixed and stained with alizarin red S. After that, the staining were extracted with 10% cetylpyridinium chloride (CPC) and subjected to spectrophotometer detection at 560 nm. MSCs in type II collagen-coated groups exhibit a higher calcium deposition level than those in the type I collagen-coated group, control group, and type I and type II mixed-coated group. The results demonstrate that type II collagen-coated PLA scaffold accelerates calcium deposition of MSCs much greater than does the commercially available PLA scaffold in osteogenic medium.

### Example 4

### The Enhanced Osteogenic Effects of the Type II Collagen-Coated Readymade Bone Materials or Implants

In this example, the osteogenic effects of type I collagen- or type II collagen-coating on hydroxyapatite-tricalcium phosphate (HA-TCP) scaffolds/implants are examined.

3D Surface Coating

3D commercial available HA-TCP bone scaffolds (herein referring to porous bone materials) are coated with purified ECM proteins (type I collagen or type II collagen) at a concentration of 5-1000 μg/ml, preferably 50-100 μg/ml, more preferably 100 μg/ml, or with 5 mM acetic acid (collagen solvent; as non-coating control) for 2 h at room temperature. After incubation, the remaining ECM solution is removed. The various bone scaffolds are further washed with PBS. The scaffolds are then air-dried in the culture hood with UV light on, and then stored at 4° C. till use.

MSCs Seeding

An aliquot of MSCs of 1×10⁵ are suspended in 175 μl of culture medium and seeded into the said HA-TCP bone scaffolds with/without coating collagens on their surfaces. After 2 hours of cell attachment in a 37° C. CO₂ incubator, the cell-loaded HA-TCP constructs (herein referring to bone implants of the present invention) are then transferred to a 24 well plate and further cultivated for designated time intervals in osteogenic medium stated above. At the end of designated time intervals, the cell-loaded HA-TCP constructs are subjected to SEM analysis.

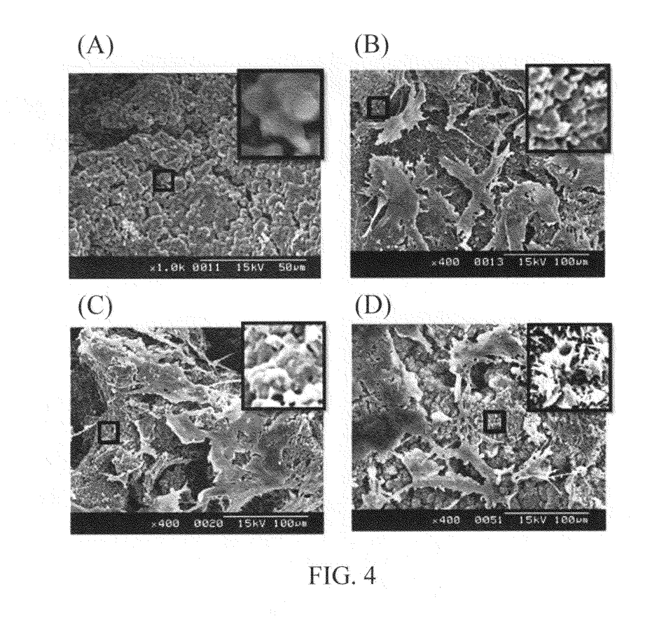

FIG. 4 shows the SEM images of the 3D cultured MSCs on non-coated (FIG. 4(B)), CI-coated (FIG. 4(C)), and CII-coated (FIG. 4(D)) HA-TCP scaffolds, and the non-cell blank (FIG. 4(A)) HA-TCP scaffold in osteogenic medium for 21 days of induction. In the figure, cells were fixed on day 21 and subjected to SEM analysis for the detection of surface ossification. The upper-right insert in each image shows the magnified calcium-deposited surface of each group at the area indicated by frames. As shown in the figure, type II collagen-coated HA-TCP showed much greater amounts of calcification crystals deposited on the implant surface than those on the other groups. This result demonstrates the enhancing effects of coated type II collagen as compared with that of type I collagen on the ossification of MSCs on 3D bone scaffolds.

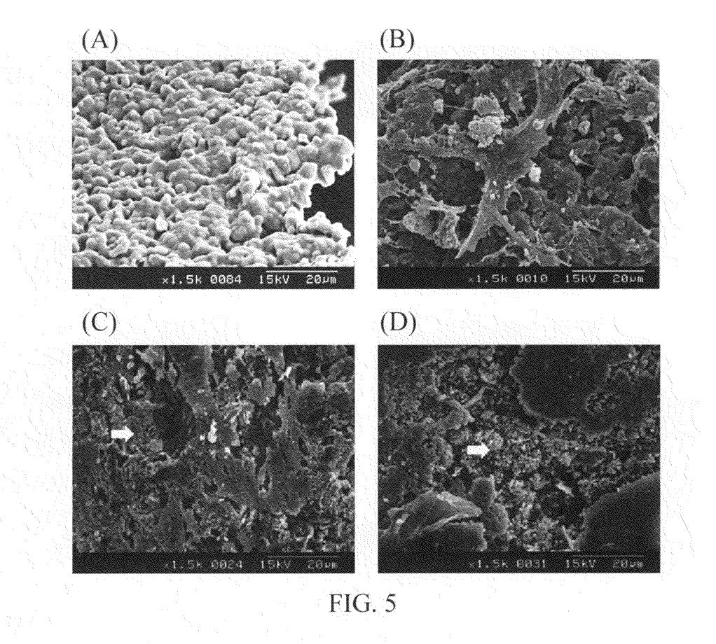

FIG. 5 illustrates the SEM images of 3D culture of MSCs on non-coated (FIG. 5(B)), CI-coated (FIG. 5(C)), and CII-coated (FIG. 5(D)) HA-TCP scaffolds, and the non-cell blank (FIG. 5(A)) HA-TCP scaffold in osteogenic medium for 42 days. After 42 days of induction, cells are fixed and subjected to SEM analysis for the detection of surface ossification. In the figure, the white arrows indicate the calcified area with mineral crystals deposited on the implant surface. As shown in FIG. 5(B), without coating collagen, the surface of the cell-loaded, non-coated HA-TCP construct shows little difference from that of the non-cell blank (FIG. 5(A)) HA-TCP scaffold. With type I collagen coating, obvious eroded HA-TCP surface and lesser extent of calcification are observed (FIG. 5(C)). With type II collagen coating, greater amounts of mineral crystals deposited on the eroded HA-TCP construct surface are observed (FIG. 5(D)). These results demonstrate the calcium deposition enhancing effect of type II collagen on MSCs cultured on HA-TCP scaffolds. It is concluded that the type II collagen enhances calcium deposition of MSCs cultured on commercial available HA-TCP scaffolds in a 3D culture condition.

The present invention provides that the type II collagen-coated surfaces or materials cause an earlier occurrence and greater level of calcium depositions than do type I collagen-coated surfaces or materials. Therefore, type II collagen not only itself could be developed into a novel form of bone repair material, but also the collagenous type II collagen-containing scaffolds could be used as better bone regenerating implants, or be coated over other readymade bone materials to generate more efficient novel bone implants. Accordingly, the applications of type II collagen on various biomaterials become a new strategy for bone regeneration especially for large bone defect repair.

While particular embodiments of the present invention have been shown and described, it will be obvious to those skilled in the art that, based upon the teachings herein, changes and modifications may be made without departing from this invention and its broader aspects. Therefore, the appended claims are intended to encompass within their scope of all such changes and modifications as are within the true spirit and scope of the exemplary embodiments of the present invention.

What is claimed is:

1. A manufacturing method of a bone implant, comprising coating or mixing at least one porous bone material with type II collagen, wherein the porous bone material is made of a material selected from the group consisting of metals, bioceramics, natural biopolymers and synthetic polymers.

2. The manufacturing method of claim 1, wherein the metal comprises titanium or titanium alloy.

3. The manufacturing method of claim 1, wherein the bioceramics comprises hydroxyapatite (HA), aluminum oxide, zirconium oxide, calcium sulfate, calcium phosphate, tricalcium phosphate, hydroxyapatite/tricalcium phosphate (HA-TCP) or a combination thereof.

4. The manufacturing method of claim 1, wherein the natural biopolymer comprises alginate, chitosan, collagen, agarose, natural extracellular matrix components, or a combination thereof.

5. The manufacturing method of claim 1, wherein the synthetic polymer comprises poly-lactic acid (PLA), poly-glycolic acid (PGA) or poly-lactic-co-glycolic acid (PLGA).

6. The manufacturing method of claim 1, wherein the type II collagen is obtained by genetic recombination of type II collagen cDNA, or by extraction and purification from a cartilage tissue of an animal comprising poultry, livestock or fishes.

7. The manufacturing method of claim 1, wherein a concentration of the type II collagen is in a range of 5 to 1000 μg/ml for coating.

8. The manufacturing method of claim 1, wherein a concentration of the type II collagen is in a range of 20 to 200 μg/ml for coating.

9. The manufacturing method of claim 1, further comprising a step of adding tissue cells selected from the group consisting of stem cells, progenitor cells and osteoblasts, wherein the stem cells and progenitor cells have a tendency toward the osteogenic differentiation.

10. The manufacturing method of claim 9, wherein the tissue cells are cultured in an osteogenic medium made of 10⁻¹⁰-10⁻⁷ M dexamethason, 5-50 mM β-glycerolphosphate, and 10-200 μg/ml ascorbic acid in Dulbecco's Modified Eagle Medium-low glucose (DMEM-LG).

11. The manufacturing method of claim 9, wherein the stem cells comprise mesenchymal stem cells obtained from bone marrow, umbilical cord blood or other somatic tissues, stem cells obtained from baby teeth or permanent teeth, or embryonic stem cells; and wherein the progenitor cells comprise mesenchymal progenitor cells obtained from bone marrow, umbilical cord blood or other somatic tissues.

12. The manufacturing method of claim 1, further comprising a step of adding a growth factor as a regulator of bone repair and regeneration.

13. The manufacturing method of claim 12, wherein the growth factor comprises bone morphogenetic protein (BMP), transforming growth factor-β (TGF-β), fibroblast growth factor (FGF), insulin-like growth factor (IGF-I), vascular endothelial growth factor (VEGF), or platelet derived growth factor (PDGF).

14. A bone implant obtained from the manufacturing method of claim 1, comprising the type II collagen and the at least one porous bone material, wherein the type II collage is coated on a surface of the porous bone material, or is mixed with the porous bone material.

15. A manufacturing method of a bone implant, comprising loading type II collagen with or without at least one bone material in a container; and lyophilizing the type II collagen to generate a type II collagen sponge construct with or without the porous bone material as the bone material.

16. The manufacturing method of claim 15, wherein a concentration of the type II collagen is in a range of 2 to 20 mg/ml.

17. The manufacturing method of claim 15, wherein the porous bone material is made of a material selected from the group consisting of metals, bioceramics, natural biopolymers and synthetic polymers.

18. The manufacturing method of claim 15, after the step of lyophilizing the type II collagen, further comprising a step of adding tissue cells selected from the group consisting of stem cells, progenitor cells and osteoblasts, wherein the stem cells and progenitor cells have a tendency toward the osteogenic differentiation.

19. The manufacturing method of claim 15, before the step of lyophilizing the type II collagen, further comprising a step of adding a growth factor as a regulator of bone repair and regeneration, wherein the growth factor comprises bone morphogenetic protein (BMP), transforming growth factor-β (TGF-β), fibroblast growth factor (FGF), insulin-like growth factor (IGF-I), vascular endothelial growth factor (VEGF), or platelet derived growth factor (PDGF).
